(19) United States
(12) Patent Application Publication
(10) Pub. No.: US 2012/0009608 A1
(43) Pub. Date: Jan. 12, 2012

(54) BIOMARKER FOR THE MONITORING AND PROGNOSIS OF CHRONIC MYELOPROLIFERATIVE DISORDERS

(75) Inventors: Barbara BOTTAZZI, Rozzano (IT); Alberto Mantovani, Rozzano (IT); Tiziano Barbui, Bergamo (IT); Alessandro Rambaldi, Bergamo (IT)

(73) Assignees: AZIENDA OSPEDALIERA OSPEDALI RIUNITI DI BERGAMO, Bergamo (IT); HUMANITAS MIRASOLE S.p.A., Rozzano (IT)

(21) Appl. No.: 12/832,608

(22) Filed: Jul. 8, 2010

(51) Int. Cl. G01N 33/543; G01N 33/566

(52) U.S. Cl. 435/7.93; 435/7.92; 435/7.94; 435/7.95; 436/501; 435/7.1

(57) ABSTRACT

The invention provides a method for monitoring the progression of a myeloproliferative disease, particularly Polycythemia Vera (PV) and Essential Thrombocythemia (ET), or the response to pharmacological treatment with JAK2 inhibitors in a patient diagnosed positive for the same disease, or a method for predicting thrombotic events in a patient affected by the same myeloproliferative diseases, based on the measurement of PTX3 concentration in a blood, plasma or serum sample.

The present invention provides methods for monitoring or prognosing chronic myeloproliferative disorders, particularly Polycythemia Vera (PV) and Essential Thrombocythemia (ET), based on the determination of PTX3 blood levels.

### INTRODUCTION

Polycythemia Vera (PV) and Essential Thrombocythemia (ET) are blood disorders which arise from the mutation and clonal expansion of a single hematopoietic stem cell(1). These diseases, classically identified as chronic myeloproliferative disorders, have been recently re-named by the World Health Organization as Myeloproliferative Neoplasms (MPN)(2). The main features of PV and ET are respectively increased red-cell mass and high platelet count. The clinical course of both PV and ET is marked by a high incidence of vascular complications, including stroke, cardiovascular events, deep vein thrombosis and pulmonary embolism, that represent the main cause of morbidity and mortality in these patients.

Cytoreduction and antithrombotic prophylaxis are recommended to reduce vascular risk however to avoid inappropriate exposure to potentially leukemogenic cytotoxic drugs, a risk-oriented therapeutic approach is advisable. Age over 60 years and prior thrombotic events identify the high-risk patients that will benefit from cytotoxic therapy while low-risk patients should be managed only with phlebotomy and aspirin. Despite the treatment, PV and ET patients still have a high risk of vascular complications, with 5-6% of affected individuals suffering for thrombotic events.

A major progress in elucidating PV and ET pathogenesis has been made with the description, in 2005, of the JAK2 somatic mutation (V617F), which is present in almost all the PV patients and in 50-60% of ET patients (3-6). Besides presence and allele burden of JAK2/V617F mutation, baseline leucocytosis has been recently recognized as a new disease-related risk factor in PV an ET (7, 8). In addition increasing evidences support the notion that leukocytosis has a prognostic significance and may be considered causative of vascular events (9). It is noteworthy that these results confirm data available in other pathological conditions, in fact it has long been known that leukocytosis and other inflammatory markers correlate with incidence of cardiovascular disease and this is consistent with the notion that atherosclerosis and thrombosis are the result of inflammatory processes.

Other inflammatory biomarkers are routinely used to assess the risk of vascular complications. One of these markers is the short pentraxin C-reactive protein (CRP), an acute phase protein produced in the liver in response to interleukin (IL)-6 (10). The introduction of high sensitive assays has permitted the routine measurement and, based on epidemiological studies, high-sensitivity CRP (hs-CRP) has been incorporated into risk assessment for cardiovascular disease. Recently a second possible marker has been identified: the long pentraxin PTX3, an acute-phase reactant considered more closely related than CRP to cardiac injuries such as myocardial infarction. PTX3 is locally produced mainly by dendritic cells, macrophages, activated leukocytes and endothelial cells in response to primary inflammatory stimuli, such as IL-1, tumor necrosis factor but not IL-6 (11). PTX3 plasma levels are significantly increased in patients with acute myocardial infarction and have prognostic value (12). The role of pentraxins has been evaluated in the Jupiter trial in which the effect of statin administration has been investigated in patients with normal lipid asset but increased plasma concentration of hs-CRP (13). The results demonstrate that reduction of hs-CRP plasma levels are associated to reduced thrombosis and reduced risk of mortality. Similarly a large study recently published demonstrate that PTX3 plasma levels are higher in patients with subclinical cardiovascular disease and are predictor of higher mortality risk (14).

Whether pentraxin plasma levels are only predictors of vascular events rather than participate in the aetiology of thrombosis is still a matter of discussion. The lack of a strict evolutionary conservation of CRP between mouse and man have precluded the use of straightforward genetic approaches to explore its in vivo function (15). In contrast PTX3 is strictly conserved in evolution and results obtained in gene-modified animals likely reflect its role in humans. Experimental data in ptx3-deficient animals demonstrate an atheroprotective and cardioprotective role of this long pentraxin (16, 17), providing the rationale to test this hypothesis in humans.

### DESCRIPTION OF THE INVENTION

The PTX3 plasma levels were analyzed in a group of patients with ET and PV, two MPNs associated with high frequency of major arterial and venous complications, to find out whether PTX3 could be a useful marker for diagnosis and monitoring of PV and ET patients. The results surprisingly demonstrate that PTX3 plasma levels are increased in the examined group of patients. It was additionally found that highest PTX3 levels correlate with JAK2/V617F allele burden and consistently, that treatment with JAK2 inhibitors down-regulate the circulating levels of PTX3. Furthermore PTX3 levels were inversely associated with the incidence of thrombotic events, providing for the first time in human pathology a support for a protective role of PTX3.

These results indicate that PTX3 is a new independent marker for monitoring Polycythemia Vera (PV) and Essential Thrombocythemia (ET) progression and that it is able to predict future major cardiovascular events in patients with myeloproliferative disorders.

In a first embodiment, the invention provides a method for monitoring the progression of PV or ET diseases or the response to pharmacological treatment with JAK2 inhibitors in a patient which has been diagnosed positive for a myeloproliferative disease, particularly PV or ET, said method comprising the following steps:
(i) providing a blood, plasma or serum sample from said patient;
(ii) measuring PTX3 concentration in that sample;
whereby an increase or diminution over time of PTX3 concentration is indicative of worsening or favourable progression of the disease state in said patient, respectively.

In a further embodiment, the invention provides a method for predicting thrombotic events in a patient affected by a myeloproliferative disease, particularly PV or ET, said method comprising the following steps:
(i) providing a blood, plasma or serum sample from said individual;
(ii) measuring PTX3 concentration in that sample;
whereby an increase or diminution over time of PTX3 concentration is indicative of a reduced or augmented risk of thrombotic events in said patient, respectively.

Based on the studies conducted by the inventors, the reference value for PTX3 concentration in healthy subjects is set in the range 0-3 ng/ml, more preferably in the range 0-2 ng/ml, but more accurate values may be determined with a larger number of individuals. Thus, in further embodiments, the above described methods comprise the additional step (iii) of comparing the measured PTX3 concentration with a reference value obtained from a statistically significant number of healthy subjects, whereby an increase or diminution over time of PTX3 concentration compared to the reference value, is indicative of the progression of the myeloproliferative disease state or of the risk of thrombotic events in said patient.

The methods for determining PTX3 concentration in a blood or plasma sample are known in the art and include immunoassays such as radio immunoassay, enzyme immunoassay, immunoprecipitation, immunostaining and Western blotting. Preferably, technologies such as direct-, indirect-, sandwich-, competitive- or multiplex-ELISA, ELISPOT assays and radioimmunoassays are used. In a typical assay, an anti-PTX3 antibody is immobilized on a solid support and a test sample is added thereon. After incubation in a suitable buffer and washing steps, the support-bound PTX3 is detected by appropriate detection means (e.g. by addition of an enzyme-labeled, secondary antibody binding the anti PTX3 antibody, thereby producing a detectable signal upon reaction with an appropriate substrate).

The preparation of anti-PTX3 antibodies is known in the art and their use to measure PTX3 levels in biological samples is described, for example, in (18).

In a further aspect, the invention provides a kit for monitoring and/or prognosing a myeloproliferative disease in a subject. The kit of the invention may include, packaged in suitable containers, antibodies against PTX3, enzyme-linked or differently labeled secondary antibodies, the biomarker PTX3 to be used as a control or to calibrate the assay, reagents and buffers.

The invention will be further illustrated in the following experimental section.

### METHODS AND RESULTS

Plasma was collected from 173 patients with ET and 71 patients with PV consecutively enrolled in the Hematology Section of Ospedali Riuniti di Bergamo. Aspirin was prescribed in the large majority of cases and chemotherapy was given in 59% and 62% of ET and PV, respectively. Mean age of patients and healthy subjects evaluated in this study are reported in Table 1.

PTX3 levels were measured in plasma collected from healthy subjects and from PV and ET patients using a sandwich ELISA based on original antibodies developed by the proponent. Protocol: ELISA plates (96 well; Nunc Immuno Plate, MaxiSorp; Nunc) were coated with 100 ng/well of rat monoclonal anti-PTX3 antibody (MNB4) diluted in coating buffer (15 mM carbonate, Na2CO3+NaHCO3, buffer pH 9.6) by overnight incubation at 4° C. Washing buffer [Dulbecco's phosphate buffered saline (PBS) containing 0.05% Tween 20] was used to extensively wash plates after each passage. Non-specific binding to the plates was blocked with 5% dry milk in washing buffer for 2 h at room temperature before adding recombinant human PTX3 standards (100 pg/ml to 2 ng/ml) or unknown samples. After incubation for 2 h at 37° C. 25 ng/well of biotin conjugated anti PTX3 rabbit IgG were then added (1 h at 37° C.) followed by addition of 100 μl of Streptavidin-peroxidase (BioSpa, Milan, Italy). Finally 100 μl of ABTS chromogen (Pierce) were added and absorbance values were read at 450 nm in an automatic ELISA reader.

Results: PTX3 plasma levels were measured following the protocol detailed above. As shown in Table 1, PTX3 median values were not different between ET and PV (3.47 ng/mL and 3.33 ng/mL respectively, P=0.888), but significantly higher than in healthy controls (1.74 ng/mL, p<0.0001).

TABLE 1

| | Controls | ET | p^{#} | PV | p^{#} |
|---|---|---|---|---|---|
| N | 32 | 173 | | 71 | |
| Age (years) | 41 (21-63) | 61 (21-96) | 0.0001 | 66 (34-94) | <0.0001 |
| median, (range) | | | | | |
| PTX3 max, | 1.74 (0.45-2.9) | 3.47 (0.8-7.75) | 0.0001 | 3.33 (0.33-10.83) | 0.0001 |
| ng/mL | | | | | |
| median, (range) | | | | | |

^{#}non parametric test between median values in healthy subject (control) and ET or PV patients.

When patients were distributed in three rounded tertiles according to PTX3 plasma levels (<2.5; 2.5-4.5; >4.5 ng/mL), a significant correlation with the amount of JAK2 allele burden was observed (p<0.01) whereas treatments were uniformly distributed in the three groups (Table 2).

TABLE 2
Variables associated with PTX3 plasma values (Univariate analysis)

| | PTX3 | | | |
|---|---|---|---|---|
| | <2.5 | 2.5-4.5 | ≧4.5 | P^{†} |
| N = 244 | 75 | 84 | 85 | |
| Age, years (median, range) | 62 (21-87) | 62 (30-96) | 66 (24-94) | 0.43 |
| Sex M/F (%) | 34/41 (45/55) | 37/47 (44/56) | 34/51 (40/60) | 0.50 |
| ET/PV (%) | 46/29 (61/39) | 68/16 (81/19) | 59/26 (69/31) | 0.28 |
| JAK2 V617F/JAK2 wild type, | 56/19 (75/25) | 58/25 (70/30) | 63/22 (74/26) | 0.94 |
| n (%) | | | | |
| If JAK2 mutated <50%/≧50% | 51/5 (91/9) | 52/6 (90/10) | 45/17 (73/27) | 0.01 |
| Any treatment, yes/no (%) | 64/11 (85/15) | 77/7 (92/8) | 78/7 (92/8) | 0.20 |
| Thrombosis | 29 (39) | 18 (21) | 21 (25) | 0.05 |

Univariate and multivariate models were used to assess the effect of PTX3 levels on thrombotic risk. As shown in Table 3, we surprisingly found that unadjusted and progressively adjusted odd ratios estimates showed an inverse association with high PTX3 levels and thrombosis, statistically significant in both the second and third tertile.

TABLE 3
Risk of thrombosis by PTX3 levels. Unadjusted and
sequentially adjusted multivariate analysis

| | 2.5 to 4.5 ng/mL | | | More than 4.5 ng/mL | | |
|---|---|---|---|---|---|---|
| | PTX3 | | | PTX3 | | |
| | OR* | 95% CI | p | OR* | 95% CI | P |
| Unadjusted | 0.43 | 0.22-0.87 | 0.019 | 0.52 | 0.26-1.00 | 0.049 |
| Adjusted for | | | | | | |
| +Age | 0.42 | 0.21-0.85 | 0.016 | 0.51 | 0.26-1.00 | 0.050 |
| +Sex | 0.41 | 0.20-0.84 | 0.015 | 0.49 | 0.25-0.98 | 0.043 |
| +Disease | 0.44 | 0.21-0.90 | 0.025 | 0.50 | 0.25-1.00 | 0.050 |
| +JAK2V617F burden | 0.47 | 0.22-0.99 | 0.046 | 0.47 | 0.24-0.98 | 0.043 |

### REFERENCES

1. Spanoudakis E, Tsatalas C. 2009. Hemopoiesis in Ph-negative chronic myeloproliferative disorders. Curr Stem Cell Res Ther 4: 154-60
2. Tefferi A, Vardiman J W. 2008. Classification and diagnosis of myeloproliferative neoplasms: the 2008 World Health Organization criteria and point-of-care diagnostic algorithms. Leukemia 22: 14-22
3. James C, Ugo V, Le Couedic J P, Staerk J, Delhommeau F, Lacout C, Garcon L, Raslova H, Berger R, Bennaceur-Griscelli A, Villeval J L, Constantinescu S N, Casadevall N, Vainchenker W. 2005. A unique clonal JAK2 mutation leading to constitutive signalling causes polycythaemia vera. Nature 434: 1144-8
4. Kralovics R, Passamonti F, Buser A S, Teo S S, Tiedt R, Passweg J R, Tichelli A, Cazzola M, Skoda R C. 2005. A gain-of-function mutation of JAK2 in myeloproliferative disorders. N Engl J Med 352: 1779-90
5. Baxter E J, Scott L M, Campbell P J, East C, Fourouclas N, Swanton S, Vassiliou G S, Bench A J, Boyd E M, Curtin N, Scott M A, Erber W N, Green A R. 2005. Acquired mutation of the tyrosine kinase JAK2 in human myeloproliferative disorders. Lancet 365: 1054-61
6. Levine R L, Wadleigh M, Cools J, Ebert B L, Wernig G, Huntly B J, Boggon T J, Wlodarska I, Clark J J, Moore S, Adelsperger J, Koo S, Lee J C, Gabriel S, Mercher T, D'Andrea A, Frohling S, Dohner K, Marynen P, Vandenberghe P, Mesa R A, Tefferi A, Griffin J D, Eck M J, Sellers W R, Meyerson M, Golub T R, Lee S J, Gilliland D G. 2005. Activating mutation in the tyrosine kinase JAK2 in polycythemia vera, essential thrombocythemia, and myeloid metaplasia with myelofibrosis. Cancer Cell 7: 387-97
7. Ziakas P D. 2008. Effect of JAK2 V617F on thrombotic risk in patients with essential thrombocythemia: measuring the uncertain. Haematologica 93: 1412-4
8. Carobbio A, Finazzi G, Guerini V, Spinelli O, Delaini F, Marchioli R, Borrelli G, Rambaldi A, Barbui T. 2007. Leukocytosis is a risk factor for thrombosis in essential thrombocythemia: interaction with treatment, standard risk factors, and Jak2 mutation status. Blood 109: 2310-3
9. Barbui T, Carobbio A, Rambaldi A, Finazzi G. 2009. Perspectives on thrombosis in essential thrombocythemia and polycythemia vera: is leukocytosis a causative factor? Blood 114: 759-63
10. Pepys M B, Hirschfield G M. 2003. C-reactive protein: a critical update. J Clin Invest 111: 1805-12
11. Garlanda C, Bottazzi B, Bastone A, Mantovani A. 2005. Pentraxins at the crossroads between innate immunity, inflammation, matrix deposition, and female fertility. Annu Rev Immunol 23: 337-66
12. Latini R, Maggioni A P, Peri G, Gonzini L, Lucci D, Mocarelli P, Vago L, Pasqualini F, Signorini 5, Soldateschi D, Tarli L, Schweiger C, Fresco C, Cecere R, Tognoni G, Mantovani A. 2004. Prognostic significance of the long pentraxin PTX3 in acute myocardial infarction. Circulation 110: 2349-54
13. Ridker P M, Danielson E, Fonseca F A, Genest J, Gotto A M, Jr., Kastelein J J, Koenig W, Libby P, Lorenzatti A J, MacFadyen J G, Nordestgaard B G, Shepherd J, Willerson J T, Glynn R J. 2008. Rosuvastatin to prevent vascular events in men and women with elevated C-reactive protein. N Engl J Med 359: 2195-207
14. Jenny N S, Arnold A M, Kuller L H, Tracy R P, Psaty B M. 2009. Associations of pentraxin 3 with cardiovascular disease and all-cause death: the Cardiovascular Health Study. Arterioscler Thromb Vasc Biol 29: 594-9
15. Hirschfield G M, Gallimore J R, Kahan M C, Hutchinson W L, Sabin C A, Benson G M, Dhillon A P, Tennent G A, Pepys M B. 2005. Transgenic human C-reactive protein is not proatherogenic in apolipoprotein E-deficient mice. Proc Natl Acad Sci USA 102: 8309-14
16. Salio M, Chimenti S, De Angelis N, Molla F, Maina V, Nebuloni M, Pasqualini F, Latini R, Garlanda C, Mantovani A. 2008. Cardioprotective function of the long pentraxin PTX3 in acute myocardial infarction. Circulation 117: 1055-64
17. Norata G D, Marchesi P, Pulakazhi Venu V K, Pasqualini F, Anselmo A, Moalli F, Pizzitola I, Garlanda C, Mantovani A, Catapano A L. 2009. Deficiency of the long pentraxin PTX3 promotes vascular inflammation and atherosclerosis. Circulation 120: 699-708
18. Peri G, Introna M, Corradi D, lacuitti G, Signorini S, Avanzini F, Pizzetti F, Maggioni A P, Moccetti T, Metra M, Cas L D, Ghezzi P, Sipe J D, Re G, Olivetti G, Mantovani A, Latini R. 2000. PTX3, A prototypical long pentraxin, is an early indicator of acute myocardial infarction in humans. Circulation 102: 636-41

1. A method for monitoring the progression of a myeloproliferative disease selected from Polycythemia Vera (PV) and Essential Thrombocythemia (ET) or the response to pharmacological treatment with JAK2 inhibitors in a patient diagnosed positive for the same disease, said method comprising the following steps:
(i) providing a blood, plasma or serum sample from said patient;
(ii) measuring PTX3 concentration in that sample;
whereby an increase or diminution over time in PTX3 concentration is indicative of worsening or favourable progression of the disease state in said patient, respectively.

2. A method for predicting thrombotic events in a patient affected by a myeloproliferative disease selected from PV or ET, said method comprising the following steps:
(i) providing a blood, plasma or serum sample from said patient;
(ii) measuring PTX3 concentration in that sample;
whereby an increase or diminution over time in PTX3 concentration is indicative of a reduced or augmented risk of thrombotic events in said patient, respectively.

3. A method according to claim 1, comprising the additional step (iii) of comparing the measured PTX3 concentration with a reference value obtained from a statistically significant number of healthy subjects, whereby an increase or diminution over time of PTX3 concentration compared to the reference value, is indicative of the progression of the myeloproliferative disease state or of the risk of thrombotic events in said patient.

4. A method according to claim 3, wherein said reference value is set in the range 0-3 ng/ml, preferably in the range 0-2 ng/ml.

5. A method according to claim 1, wherein PTX3 concentration is measured with one of following techniques: direct ELISA, indirect ELISA, sandwich ELISA, competitive ELISA, multiplex ELISA, ELISPOT or radioimmunoassay.

6. A method according to claim 2, comprising the additional step (iii) of comparing the measured PTX3 concentration with a reference value obtained from a statistically significant number of healthy subjects, whereby an increase or diminution over time of PTX3 concentration compared to the reference value, is indicative of the progression of the myeloproliferative disease state or of the risk of thrombotic events in said patient.

7. A method according to claim 6, wherein said reference value is set in the range 0-3 ng/ml, preferably in the range 0-2 ng/ml.

8. A method according to claim 2, wherein PTX3 concentration is measured with one of following techniques: direct ELISA, indirect ELISA, sandwich ELISA, competitive ELISA, multiplex ELISA, ELISPOT or radioimmunoassay.
